(19) United States
(12) Patent Application Publication
(10) Pub. No.: US 2007/0250041 A1
(43) Pub. Date: Oct. 25, 2007

(54) Extendable Interventional Medical Devices

(76) Inventor: Peter R. Werp, St. Louis, MO (US)

Correspondence Address: Bryan K. Wheelock, Suite 400, 7700 Bonhomme, St. Louis, MO, 63105, US

(21) Appl. No.: 11/737,357

(22) Filed: Apr. 19, 2007

Related U.S. Application Data
(60) Provisional application No. 60/793,027, filed on Apr. 19, 2006.

(51) Int. Cl. A61M 25/098

(52) U.S. Cl. 604/529

(57) ABSTRACT

An elongate medical device is provided that may be magnetically navigated through a subject's body, which includes an extendable and retractable tip element disposed on the distal end of the medical device. The tip element is configured to be retracted prior to advancing the distal end of the medical device towards a target area within the subject's body, and to be controllably extended towards the target area within the subject's body. An actuation means is provided for controllably extending the tip element, which enables fine control of the advancement of the tip of the medical device towards a target location within a subject's body. The actuation means may be achieved through hydraulic pressure application to an expandable volume, electrical current application to a material that responsively changes length, electrical current application to an electromagnet for responsively repelling and displacing a magnet, and mechanical force application for displacing a spring-loaded mechanism.

### CROSS-REFERENCE TO RELATED APPLICATIONS

This application claims benefit of U.S. Provisional Patent Application Ser. No. 60/793,027, filed Apr. 19, 2006, the entire disclosure of which is incorporated by reference.

### FIELD

The present disclosure relates to devices and methods for interventional medicine, and more specifically to navigation of medical devices through the body to an operating region.

### BACKGROUND

Interventional medicine is the collection of medical procedures in which access to the site of treatment is made through one of the patient's blood vessels, body cavities or lumens. Interventional medicine technologies have been applied to the manipulation of instruments which contact tissues during surgical procedures. Several presently available interventional medical systems for navigating an interventional medical device through a subject's lumens direct and orient the device distal tip by means of a navigation mechanism, such as magnetic navigation, using computer assisted navigation and an imaging system to provide real-time imaging of the device and blood vessels and tissues. Such systems can control the navigation of a medical device, such as a catheter, to a target destination in an operating region using a computer and controlled navigation mechanism to orient and guide the distal tip through blood vessels and tissue. To reach the target destination, a navigation system must accurately control the device tip as it approaches the target before advancing the remaining distance to reach the given target. In some cases, the device tip may not reach the desired target due to inaccuracies in the system or due to difficulties in navigating the device.

### SUMMARY

Embodiment of the present invention provides for controllably extending and retracting the distal end of a medical device that is adapted to be magnetically navigated within a subject's body. In one aspect of the present invention, a magnetically navigable medical device is provided that has a proximal end, an elongated lumen, and a distal end having an extendable and retractable tip. The distal tip element is configured to be retracted prior to advancing the distal end of the medical device near to a target area within the subject's body, and to be controllably extended towards the target area within the subject's body. An actuation means is provided for controllably extending the tip element, which enables fine control of the advancement of the tip of the medical device towards a target location within a subject's body. The actuation means can be selected from the group comprising hydraulic pressure application to an expandable volume, electrical current application to a material that responsively changes length, electrical current application to an electromagnet for responsively repelling and displacing a magnet, and mechanical force application for displacing a spring-loaded mechanism. The actuation means is either under control of a physician, or under computer control for automatic extension and retraction. In either case, feedback information is available to the user or computer in the form of real-time imaging or real-time positioning of the device distal tip with respect to the subject anatomy. Additional feedback information of use in navigation in specific embodiments include data from an ultrasound probe, contact monitoring probe, or force-sensing probe, all such probes being located at or near the distal tip element.

In another aspect of the present invention, a method for controllably advancing a medical device having a retractable and extendable tip element is provided. The method provides for controllably advancing the medical device towards a target area within a subject's body, whereby the method includes introducing the distal end of the medical device into a subject's body, and navigating the distal end towards a target area within the subject's body. The method provides for retracting the retractable tip element prior to advancing the distal end of the medical device near the target area within the subject's body, and controllably extending the tip element toward the target area. Extending the retractable and extendable tip a minute distance towards a target area can be finely controlled and achieved by at least one actuation means under physician or computer control. The fine control of the retractable and extendable tip element can be achieved by controlling an actuation means selected from the group comprising hydraulic pressure application to an expandable volume, electrical current application to a material that responsively changes length, electrical current application to an electromagnet for responsively repelling and displacing a magnet, and mechanical force application for displacing a spring mechanism, to retract and extend a tip element.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

FIG. 1-A presents a block diagram of an interventional system for use of a magnetically navigable device having an extendable and retractable tip element according to the principles of the present invention;

FIG. 1-B shows the extendable tip element near target tissue in the region of intervention;

FIG. 2 is a flow-chart of the navigation process for two specific applications;

FIG. 3 shows a cut-away side elevation view of one embodiment of a medical device according to the principles of the present invention with a hydraulic actuation mechanism;

FIG. 4 is a cut-away side elevation view of a second embodiment of a medical device according to the principles of the present invention with electro-magnetic actuation;

FIG. 5 is a cut-away side elevation view of a third embodiment of a medical device according to the principles of the present invention with electrostrictive actuation;

FIG. 6 is a cut-away side elevation view of a fourth embodiment of a medical device according to the principles of the present invention with mechanical actuation;

FIG. 7 illustrates application of a device according to the principles of the present invention to the crossing of chronic total occlusions; and

FIG. 8 illustrates application of a device according to the principles of the present invention to the diagnosis and treatment of heart conditions.

Corresponding reference numerals indicate corresponding parts throughout the several views of the drawings.

### DETAILED DESCRIPTION OF THE INVENTION

The following description is merely exemplary in nature and is not intended to limit the present disclosure, application, or uses. It should be understood that throughout the drawings, corresponding reference numerals indicate like or corresponding parts and features.

In the various embodiments, a navigable elongated medical device having a proximal end and a distal end is provided that is adapted to be navigated through a subject's body, and to be controllably extended towards a target area within the subject's body. The elongated medical device includes an extendable and retractable tip element that is disposed on the distal end of the medical device. The extendable and retractable tip element may be finely controlled to extend the tip a minute distance, for controlling the approach of the medical device towards a target area. The medical device is adapted to be inserted into a subject's vasculature and navigated towards a target destination, such as the heart for example. The extendable tip element of the medical device is adapted to be retracted, and is preferably in a retracted state prior to advancing the distal end of the medical device to the target area. Where the elongate medical device has encountered several turns through the subject's body during navigation towards a target area, the degree of advancement control may be greatly reduced. To advance the tip with finesse, the extendable tip element is adapted to be controllably extended. The tip element may be finely controlled to extend the tip towards a target area within the subject's body that may be difficult to locate. Examples of such situations may include a tiny side vessel of the vasculature, or when the tip must be extremely near or in contact with moving heart wall tissue. In the various embodiments, the tip element is controllably contracted or extended by an actuation means selected from the group consisting of hydraulic pressure application to an expandable volume, electrical current application to a material that responsively changes length, electrical current application to an electromagnet for responsively repelling and displacing a magnet, and mechanical force application for displacing a spring mechanism.

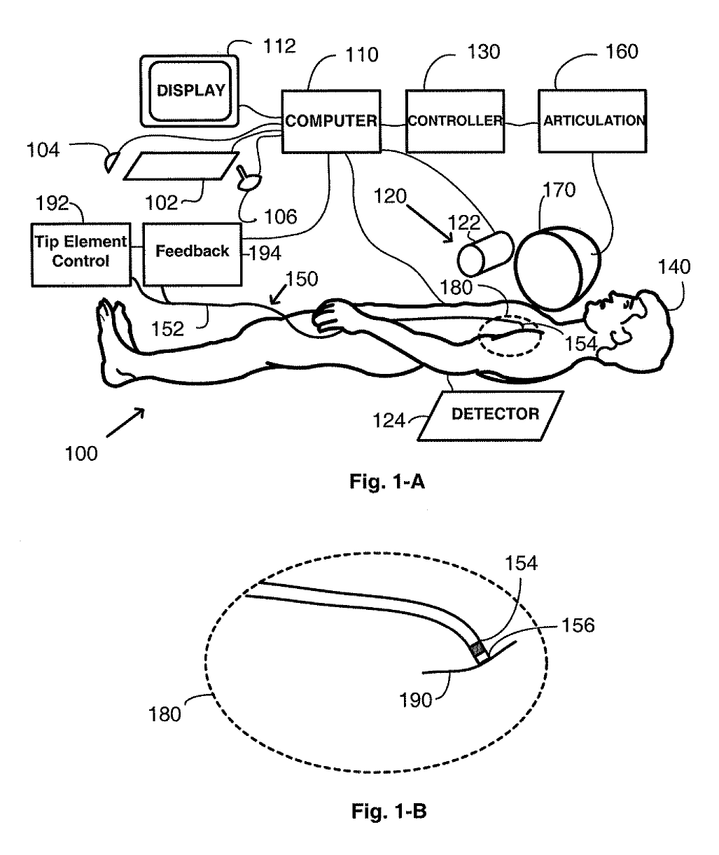

One embodiment of a medical device 150 having a proximal end 152 and a distal end 154 is provided for use in an interventional system 100 is shown in FIG. 1-A. A subject 140 is positioned within the interventional system, and the medical device is inserted into a blood vessel of the subject and navigated to an intervention region or volume 180. In magnetic navigation a magnetic field externally generated by magnet(s) 170 orients a small magnetically responsive element, which is preferably a magnet, located at the device distal end (not shown). Real time information is provided to the physician, for example by an x-ray imaging chain 120 comprising an x-ray tube 122 and an x-ray detector 124, and also possibly by use of three-dimensional device localization system such as a set of RF emitters located at the device distal end (not shown) or similar localization device. The physician provides inputs to the navigation system through a user-interface computer 110 comprising a display system 112, a keyboard 102, mouse 104, joystick 106, and similar input devices. Display 112 also shows real-time image information acquired by the imaging chain 120. Computer 110 relays inputs from the user to a controller 130 that determines the magnet(s) orientation through articulation control 160.

As shown in FIG. 1-B, once the device distal end 154 has reached the region of operation, the device tip element 156 is extended to make contact with the tissue of interest 190 for acquisition of diagnostic parameters, such as electric signals within the heart tissues, or for the treatment of specific conditions, such as tissue ablation in the treatment of cardiac arrhythmia. Device tip 156 is preferably dynamically extendable and retractable, for example to allow dynamic tracking of the heart wall motion to maintain adequate wall pressure while preventing application of excessive force that could lead to catastrophic tissue puncture. Controller 192 is in communication with computer 110, and also with the physician through the user interface previously described. The controller 192 controls the extension and retraction of the device tip element.

In specific embodiments, device tip 156 can have sensor(s), such as strain gauges or similar devices located at or near the device tip to provide force data information to estimate the amount of pressure applied on the target tissue, as feedback to system 100 in determining the device tip extension or retraction; other sensors might include an ultrasound device or other device appropriate for the determination of distance from the device tip to the tissue. Feedback data from the tip element and the device distal end are processed by feedback block 194 which in turns communicates with the tip element control block 192 as well as with computer 110. Further device tip feedback data can include relative tip and tissues positions information provided by an imaging system, predictive device modeling, or device localization system. In closed loop implementation, the device tip control 192 provides input commands to the device tip actuation mechanism based on feedback data and previously provided input instructions; in semi-closed loop implementations, the physician also contributes to the navigation, based in part upon feedback data. Control commands and feedback data may be communicated from the user interface and control 192 to the device and from the device tip back to the feedback block 194, through cables or other means, such a wireless communications and interfaces. As known in the art, control block 192 comprises an electromechanical device advancer (not shown), capable of precise device advance and retraction based on corresponding control commands.

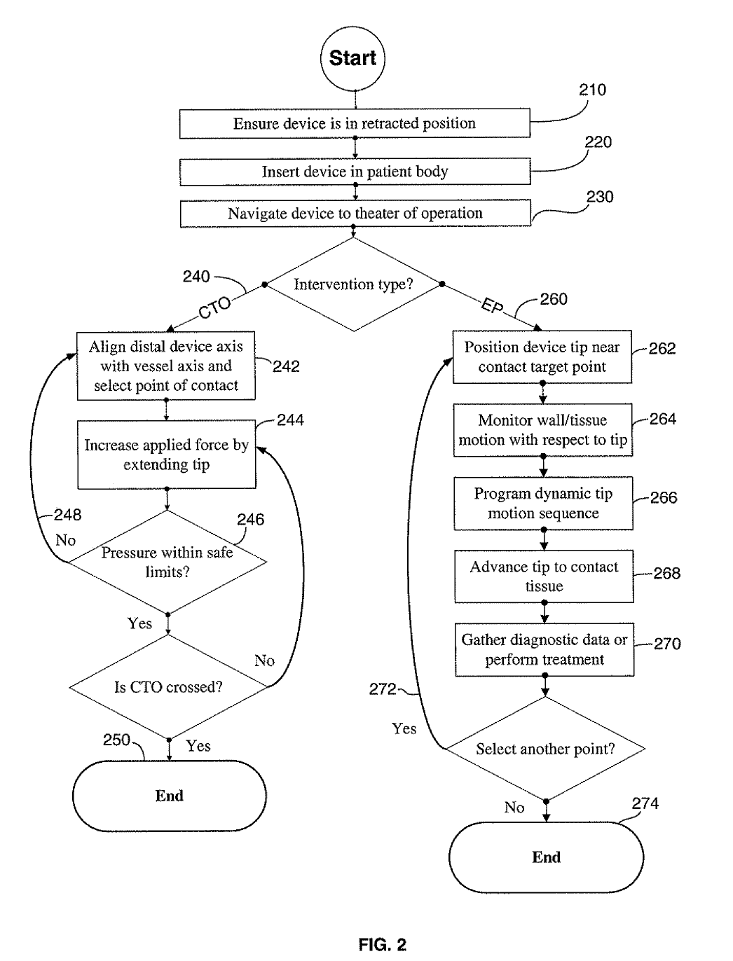

In another aspect of the invention, a method is disclosed that enables magnetic navigation of an interventional device to a region of operation and subsequent acquisition of diagnostic information and/or treatment of specific conditions. FIG. 2 provides a flow-chart for two embodiments of the method. At the start, and with the device tip in retracted position, 210, the device tip is inserted within the patient's body, 220. Using magnetic navigation, the device distal end is navigated to a region of operation, 230. Depending on the intervention type, different step sequences in the method ensue. If the intervention is for the treatment of a chronic total occlusion (CTO), 240, the device distal end is aligned such that its local axis is essentially aligned with the local vessel axis, and a point of contact on the occlusion is selected, 242. In the following step, 244, the pressure on the contact point is increased by extending the device distal tip. The pressure is monitored, 246, and if determined to be at the limit of safe practice, another contact point is selected, 248, and the method iterated. If not, the applied pressure is increased till it suddenly drops, indicating that the CTO has been successfully crossed, 250.

If the intervention is for the acquisition of diagnostic heart information, such as in the case of planned cardiac tissue ablation for the treatment of arrhythmia in electro-physiology (EP) applications, 260, the device tip is positioned in the neighborhood of the heart wall tissue to be evaluated or treated, 262. The quasi-periodic motion of the heart wall is monitored, for example using ultrasound technology, 264, and a corresponding dynamic tip extension/retraction sequence is programmed for the device, 266. The tip is then advanced to contact the tissue with appropriate force, 268, and upon contact (as determined, for example, from a contact sensor measuring electrical currents), the quasi-periodic motion sequence is activated with the appropriate phase to match the tissue motion. If necessary, adjustments are made to the programmed quasi-periodic tip sequence so that contact pressure is maintained and remains within safe values. Then, diagnostic data are collected or treatment, such as tissue ablation, is performed, 270. Once this step is completed, the method iterates to the next selected contact point, 272, or terminates, 274. Even though only two specific applications are illustrated in this method flow-chart, other interventions are possible by application of the disclosed method.

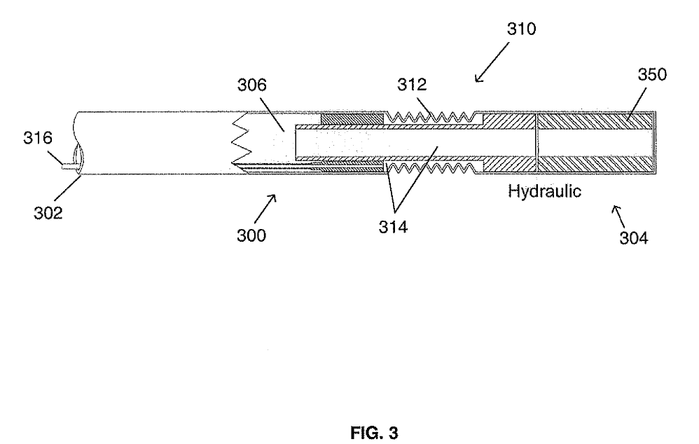

In a first device embodiment as shown in FIG. 3, the navigable medical device 300 comprises a proximal end 302, a distal end generally indicated by numeral 304, and a lumen 306 therebetween. The navigable medical device further includes a tip element 310 disposed on the distal end 304. The tip element 310 includes a magnetically responsive or permanent magnet element 350 at its distal most end. The tip element 310 comprises a plurality of annular folds 312 defining a space 314 that extends and retracts longitudinally with changes in pressure. The tip element 310 is controllably extended and retracted by controlling the level of hydraulic fluid pressure applied to the space 314, which may be communicated through a fluid supply line 316 from the proximal end 302 of the medical device 300. The plurality of annular folds 312 defining the space 314 are preferably a bellows that expands and contracts as the space expands longitudinally relative to the medical device. The bellows provide a spring-like function, and would normally hold the tip 310 in a retracted state when the hydraulic fluid pressure within the space 314 is at a minimum. If necessary the hydraulic pressure acts against an additional spring element (not shown). Increasing the hydraulic fluid pressure to the space 314 would longitudinally expand the bellows to extend the tip element 310. Accordingly, by utilizing a hydraulic fluid medium at the proximal end 302 and controlling the application of a hydraulic fluid pressure communicated through a supply line 316 to the space 314 in the tip 310, fine control of tip extension is provided.

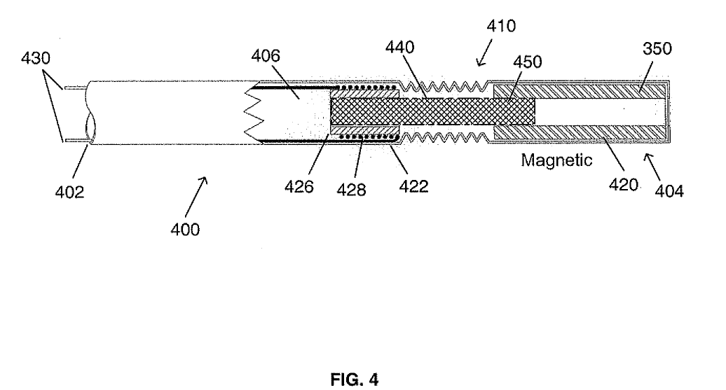

In a second device embodiment as shown in FIG. 4, the navigable medical device 400 comprises a proximal end 402, a distal end generally indicated by numeral 404, and a lumen 406 therebetween. The navigable medical device further includes a tip element 410 disposed on the distal end 404. The tip element 410 comprises a permanent magnet tip 420 and an electromagnet 422 that causes the permanent magnet tip 420 to be variably displaced from the electromagnet 422 as a function of the current through the electromagnet 422. The electromagnet 422 comprises a coil support element 426 having a conductive wire coil 428 wound about it. The permanent magnet tip 420 slides along longitudinal element 440 and is controllably extended and contracted by controlling the current level through the electromagnet 422. An electrical current may be conducted to the coil 428 of the electromagnet 422 via a pair of wires 430 that extend from the proximal end 402 through the lumen 406 to the electromagnet 422. The permanent magnet tip 420 is attracted to or repulsed from the electromagnet 422 depending on the direction of current through the electromagnet 422. Current conducted through the electromagnet 422 of a specific polarity causes the permanent magnet 420 to be repelled or displaced from the electromagnet 422 as a function of current intensity to the electromagnet 422. The tip element 410 may further comprise a spring disposed between the permanent magnet tip 420 and the electromagnet 422 for biasing the permanent magnet 420 away from the electromagnet 422, to aid in displacing the permanent magnet 420. Accordingly, by controlling an electrical current source at the proximal end 402 and conducting the current communicated via wires 430 through the lumen 406 to the electromagnet 422, fine control over the extension or retraction of the tip element 410 is provided. In a second embodiment the electromagnet 422 coil support element 426 comprises a magnetically permeable material that provides a retracted bias to the extendable tip element by attracting the permanent magnet element 350.

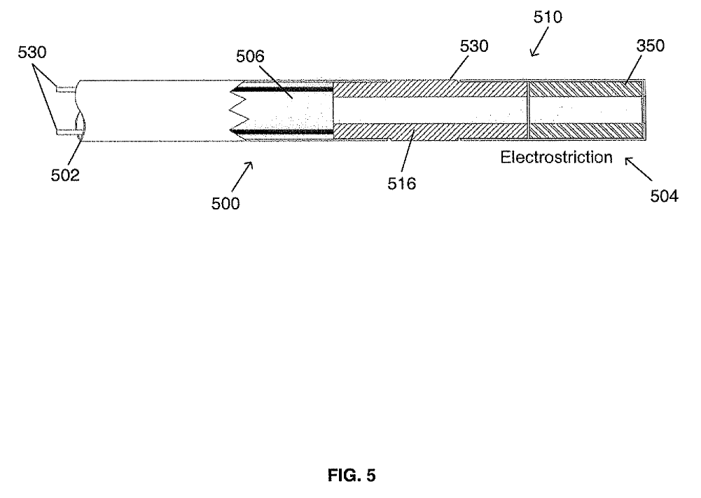

In a third device embodiment as shown in FIG. 5, the navigable medical device 500 comprises a proximal end 502, a distal end generally indicated by numeral 504, and a lumen 506 therebetween. The navigable medical device further includes a tip element 510 disposed on the distal end 504. The tip element 510 includes a magnetically responsive or permanent magnet element 350 at its distal most end. The tip element 510 further comprises an electrostrictive element 516 that changes length as a function of a voltage applied to the electrostrictive element 516, and the tip element 510 is controllably extended and contracted by controlling the voltage applied to the electrostrictive element 516. A voltage may be applied to the electrostrictive element 516 via a pair of wires 530 that extend from the proximal end 502 through a lumen 506 to the electrostrictive element 516. The electrostrictive element is 516 made of a polymer that varies in length as a function of an applied voltage, wherein the length may be finely controlled by varying the voltage level to the electrostrictive element 516. Accordingly, by controlling a voltage source at the proximal end 502 and applying the voltage communicated via wires 530 through the lumen 506 to the electrostrictive element 516, fine control over expansion of the tip 510 is provided. Additionally the electrostrictive element may work against a spring (not shown).

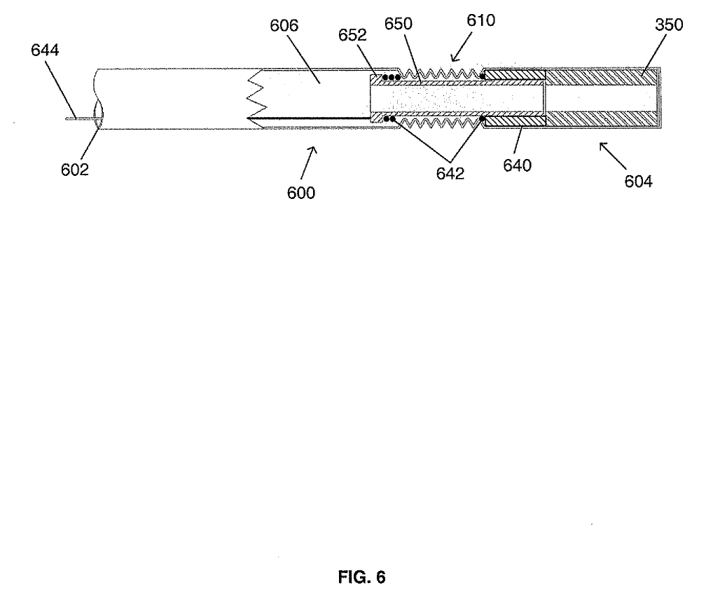

In a fourth device embodiment as shown in FIG. 6, the navigable medical device 600 comprises a proximal end 602, a distal end generally indicated by numeral 604, and a lumen 606 therebetween. The navigable medical device further includes a tip element 610 disposed on the distal end 604 shown in retracted position. The tip element 610 includes a magnetically responsive or permanent magnet element 350 at its distal most end. The tip element 610 utilizes the application of a mechanical force for displacing a spring-like mechanism 642 (only a few spring turns shown). The tip element 610 comprises an end part 640 that can slide with respect to fixed longitudinal element 650. Longitudinal element 650 comprises an abutment 652 against which spring mechanism 642 acts to extend tip element against retaining force applied through at least one pull wire 644 extending from the proximal end 602 of the medical device 600 to the distal end 640. The end element 610 may be controllably extended and contracted by controlling the force applied to the wire 644 for controlling the extension of the retractable section 640. Accordingly, by controlling the application of a force at the proximal end 602 to at least one wire 644 extending through the lumen 606 to the retractable and extendable section 640, fine control over the length of the retractable and extendable section 640, and thereby the extension of the tip 610 element is provided.

In the various embodiments of a medical device described above, the tip element further comprises at least one magnetically responsive element 350 disposed in the distal end of the medical device. The magnetically responsive element 350 can be made of a permanent magnetic material or a permeable magnetic material, and is configured to provide for magnetic navigation of the distal end of the medical device. In the presence of an applied magnetic field, the distal end of the medical device will tend to align with the field direction to the extent allowed by the flexibility of the medical device. The magnetically responsive element 350 is of sufficient size and shape to cause the distal end of the medical device to align in a selected direction with a magnetic field applied from an external source magnet. Suitable permanent magnetic materials include neodymium-iron-boron (Nd—Fe—B), Suitable permeable magnetic materials include magnetic stainless steel, such as a 303 or 304 stainless steel, or other alloys such as Hiperco. Permeable magnetic materials may be used as a substitute for but preferably in combination with permanent magnetic materials. The size and material of the magnetically responsive element 350 are selected so that the distal end of the guide wire can be reoriented by the application of a magnetic field of no more than about 0.10 Tesla, and more preferably no more than about 0.08 Tesla, and more preferably no more than about 0.06 Tesla. In the preferred embodiment, the length of the magnetically responsive element 350 is preferably at least 1.0 millimeter, but may alternatively be any length in the range of 0.5 to 5 millimeters.

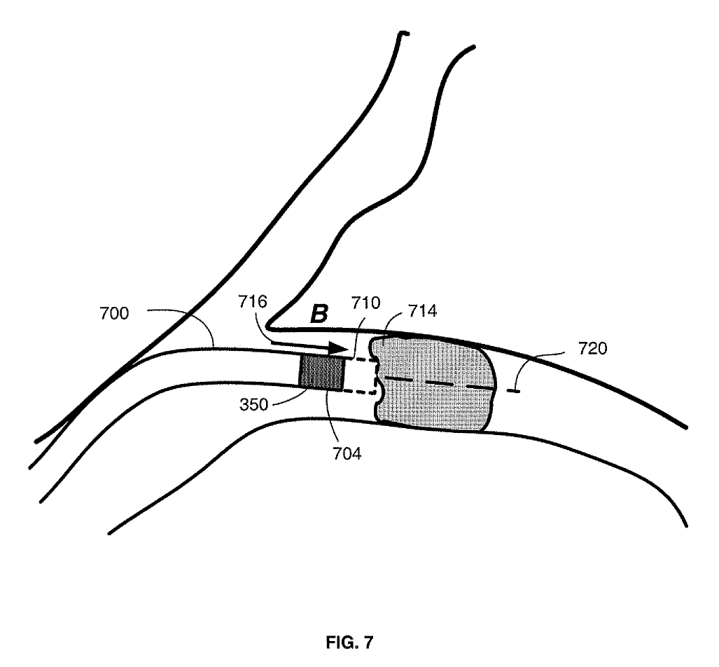

FIG. 7 shows application of a device according to the principles of the present invention to the treatment of chronic total occlusion. Medical device 700 comprises a distal end 704 that is navigated to the neighborhood of an occlusion 714. The externally generated magnetic field 716 pulls the tip magnet 350 in the direction of the occluded vessel axis 720. The extendable and retractable tip element 710 further comprises an ablation element, such as an RF antenna, optical ablation device, mechanical burr, or other device suitable for creating blood passageway trough occlusion 714. The tip element is extended to contact the occlusion during treatment. A combination of proximal device advance and magnetic navigation ensures that the tip orientation remains in alignment with local vessel axis as the device progresses through the occlusion.

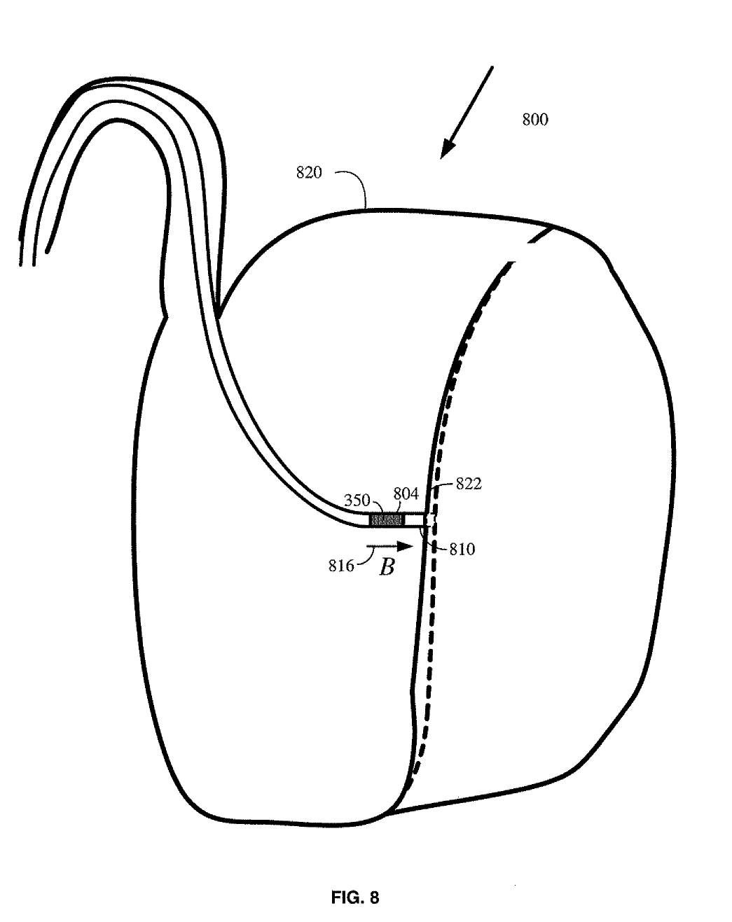

FIG. 8 schematically describes 800 the use of a device according to the principles of the present invention in EP applications. An elongated medical device comprising a distal end 804 is navigated to a cardiac chamber, for example right cardiac atrium 820. The extendable device tip 810 is positioned near the myocardial wall 822 within reach of the extendable tip 810 and maintained in the neighborhood of that position through a combination of automated proximal device advance and magnetic field pull on tip device magnet element 350 through externally generated magnetic field B 816. For a given target point on the heart wall, the distance from the device tip to the wall is measured through the quasi-periodic wall cycle. A corresponding extension/retraction sequence is programmed for the tip extension so as to ensure that contact between the device tip and myocardial wall is maintained while applied pressure remains below a safe level to preclude catastrophic tissue puncture. Adjustments to the automated tip extension and retraction sequence may be made on the basis of feedback signal from distance and/or pressure sensors located at the device tip (not shown). Upon completion of the diagnostic data collection, as in patterns of currents, or of the therapeutic treatment, as in the ablation of wall tissues, the procedure iterates for the next target point.

In another aspect of the present invention, organ motion tracking is achieved by dynamically retracting and advancing the entire device from its proximal end. Given a known device length inserted in the subject's body, and modeled transfer function relating input advancer increments to device tip travel distances, a sequence of advancer commands is programmed into device control block 192. Adjustments to the sequences are made based on feedback inputs from the device tip to the feedback processing block 194 and control block 192. In such a way, dynamic tracking of an organ is achieved by proximally advancing and retracting the entire device inserted length, without need for a separate extendable tip element.

Dynamic and adaptive organ wall motion tracking can also be achieved by other means. In one embodiment of the present invention, a device is provided with a set of pull-wires, extending from the device proximal end to various wire termination points along the device length, as known in the art. The predicted wall motion is processed by control block 192, and sequences of pull-wire retractions and releases are programmed into pull-wire servo-motors. Adjustments to the sequences are made based on feedback inputs from the device tip to the feedback processing block 194 and control block 192. In such a way, dynamic tracking of an organ is achieved by relying on the pull action of the wires acting against the device mechanical flexibility and associated recoil behavior, without need for a separate extendable tip element. Dynamic pull-wire activation sequences can be combined with dynamic proximal device advances and retraction, thereby permitting organ motion tracking with increased flexibility and over larger motion ranges than possible with either of these two approaches separately.

Either one of the embodiments just described, using proximal device advance and retraction, pull-wire activation sequences, or combination thereof, can be combined with a separately actuated device tip element extension and retraction, to achieve tracking of an organ motion over distances that might not otherwise be achievable.

These various mechanical embodiments of a device allowing organ motion tracking can also be combined with magnetic navigation. Magnetic navigation enables finer control of the device distal tip by ensuring that contact is maintained or repeated within a small area of the tissue, typically within a millimeter. Magnetic navigation also enables small controlled dynamic adjustments that might be difficult to achieve by mechanical means only, thereby providing quick response to changes in body parameters, such as heart rate, or changes in local blood flow patterns.

Although the present invention has been described with respect to several exemplary embodiments, there are many other variations of the above-described embodiments that will be apparent to those skilled in the art, even where elements have not explicitly been designated as exemplary. It is understood that these modifications are within the teaching of the present invention, which is to be limited only by the claims appended hereto.

What is claimed is:

1. A magnetically navigable medical device having a proximal end and a distal end adapted to be navigated through a subject's body, the device comprising an extendable and retractable tip element disposed on the distal end.

2. The device of claim 1 wherein the extendable tip element is adapted to be contracted prior to advancing the distal end of the medical device near a target area within the subject's body, and is adapted to be extended towards a target area within the subject's body.

3. The device of claim 1 wherein the tip element is activated by an actuation means selected from the group consisting of hydraulic pressure application to an expandable volume, electrical current application to a material that responsively changes length, electrical current application to an electromagnet for responsively repelling and attracting a magnet, and mechanical force application for displacing a spring mechanism.

4. The device of claim 3 wherein the actuation means is controlled by a control means allowing fine adjustments to the tip element extension.

5. The device of claim 3 wherein the tip element comprises a plurality of annular folds defining a volume that expands and contracts longitudinally with changes in pressure, and the tip element is controllably extended and contracted by controlling the level of hydraulic fluid pressure applied to the volume.

6. The device of claim 5 wherein the plurality of annular folds defining a volume is a bellows that expands and contracts with changes in hydraulic pressure within the bellows.

7. The device of claim 3 wherein the tip element comprises an electrostrictive element that changes length as a function of applied voltage and is controllably extended and retracted by controlling the voltage applied to the electrostrictive element.

8. The device of claim 7 wherein the electrostrictive element is made of a polymer that varies in length as a function of an applied voltage, wherein the length may be finely controlled by varying the voltage level to the electrostrictive element.

9. The device of claim 3 wherein the tip element comprises a permanent magnet tip and an electromagnet that causes the permanent magnet tip to be variably displaced from the electromagnet as a function of the current through the electromagnet, and the permanent magnet tip is controllably extended and contracted by controlling the current level through the electromagnet.

10. The device of claim 9 wherein the electromagnet further comprises a permeable magnetic material and the permanent magnet tip is attracted to the electromagnet in the absence of current through the electromagnet, which attraction is reduced when current is conducted through the electromagnet to cause the permanent magnet to be repelled from the electromagnet as a function of the current to the electromagnet.

11. The device of claim 9 wherein the tip element further comprises a spring disposed between the permanent magnet tip and the electromagnet for biasing the permanent magnet away from the electromagnet.

12. A magnetically navigable medical device having a proximal end and a distal end adapted to be navigated and advanced through a subjects body, the navigable medical device comprising:
an extendable and retractable tip element disposed on the distal end; and
an actuation means for controllably extending and retracting the tip element, the actuation means being selected from the group consisting of hydraulic pressure application to an expandable volume, electrical current application to a material that responsively changes length, electrical current application to an electromagnet for responsively repelling and displacing a magnet, and mechanical force application for displacing a spring mechanism.

13. The device of claim 12, wherein the tip element comprises a plurality of annular folds defining a volume that expands and contracts longitudinally with changes in pressure, the tip element being controllably extended and retracted by controlling the level of hydraulic fluid pressure applied to the volume.

14. The device of claim 13 wherein the plurality of annular folds defining a volume is a bellows that expands and contracts with changes in hydraulic pressure within the bellows.

15. The device of claim 12 wherein the tip element comprises an electrostrictive element that changes length as a function of a voltage applied to the electrostrictive element, and the tip element is controllably extended and retracted by controlling the voltage applied to the electrostrictive element.

16. The device of claim 15 wherein the electrostrictive element is made of a polymer that varies in length as a function of an applied voltage, wherein the length may be finely controlled by varying the voltage level to the electrostrictive element.

17. The device of claim 12 wherein the tip element comprises a permanent magnet tip and an electromagnet that causes the permanent magnet tip to be variably displaced from the electromagnet as a function of the current through the electromagnet, and the permanent magnet tip is controllably extended and retracted by controlling the current level through the electromagnet.

18. The device of claim 17 wherein the electromagnet comprises a permeable magnet, and the permanent magnet tip is attracted to the electromagnet in the absence of current through the electromagnet, which attraction is reduced when current is conducted through the electromagnet to cause the permanent magnet to be repelled from the electromagnet as a function of the current to the electromagnet.

19. The device of claim 17 wherein the tip element further comprises a spring disposed between the permanent magnet tip and the electromagnet for biasing the permanent magnet away from the electromagnet.

20. The device of claim 12 wherein the tip comprises an end section in connection with a retractable and extendable section, a spring for biasing the end section towards an extended position, and a wire extending from the proximal end of the medical device to the retractable and extendable section for displacing the spring, the end section being controllably extended and retracted by controlling the force applied to the wire for displacing the spring to retract the retractable and extendable section.

21. A method for controllably advancing a medical device having a retractable and extendable tip element towards a target area within a subject's body, the method comprising:
introducing the distal end of the medical device into a subject's body;
magnetically navigating the distal end towards a target area within the subject's body; and
controllably extending the retractable and extendable tip a distance towards a target area within the subject's body.

22. The method of claim 21, further comprising retracting the retractable and extendable tip prior to insertion in the subject's body.

23. The method of claim 21 wherein the controllably extending the retractable and extendable tip is achieved by a controlling an actuation means selected from the group consisting of hydraulic pressure application to an expandable volume, electrical current application to a material that responsively changes length, electrical current application to an electromagnet for responsively repelling and displacing a magnet, and mechanical force application for displacing a spring mechanism to retract and extend a tip element.

24. A method of dynamically moving the distal end of an interventional device, the method comprising:
a) providing an interventional device comprising at least one component from the group consisting of i) pull-wires; ii) electromechanical proximal advancer; and iii) an extendable and retractable device distal tip element; and
b) controllably actuating at least one component of interventional device (a) to track an organ motion.

25. The method of claim 24, further comprising adjusting actuation based on predicted organ motion.

26. The method of claim 24, further comprising adjusting actuation based on device tip feedback data.

27. A method of dynamically moving the distal end of a magnetically navigable interventional device, the method comprising:
a) providing an interventional device comprising at least one tip magnet and further comprising at least one component from the group consisting of i) pull-wires; ii) electromechanical proximal advancer; and iii) an extendable and retractable device distal tip element;
b) magnetically controlling the device progress during at least part of the navigation; and
c) controllably actuating at least one component of interventional device (a) to track an organ motion.

28. The method of claim 27, further comprising adjusting actuation based on predicted organ motion.

29. The method of claim 27, further comprising adjusting actuation based on device tip feedback data.

30. The method of claim 27, further comprising adjusting magnetic navigation based on device tip feedback data.
